(19) United States
(12) Patent Application Publication
(10) Pub. No.: US 2024/0392223 A1
(43) Pub. Date: Nov. 28, 2024

(54) OPEN MICROFLUIDIC TISSUE CULTURE SYSTEM

(71) Applicant: University of Washington, Seattle, WA (US)

(72) Inventors: Amanda Haack, Kenmore, WA (US); Ashleigh Brooks Theberge, Seattle, WA (US); Lauren Brown, Seattle, WA (US); Erwin Stefan Peter Berthier, Seattle, WA (US); Cole Alexander DeForest, Seattle, WA (US); Nathan J. Sniadecki, Bothell, WA (US); Serena Nguyen, Seattle, WA (US); Priti Mulimani, Seattle, WA (US); Alex Goldstein, Seattle, WA (US); Thomas P. Leahy, Seattle, WA (US); Ross Bretherton, Seattle, WA (US); Asha R. Viswanathan, Seattle, WA (US); Ariel Lin, Seattle, WA (US); Jamison Whitten, Seattle, WA (US)

(73) Assignee: University of Washington, Seattle, WA (US)

(21) Appl. No.: 18/669,367

(22) Filed: May 20, 2024

Related U.S. Application Data
(60) Provisional application No. 63/503,677, filed on May 22, 2023.

(51) Int. Cl. C12M 3/06; C12M 1/12; C12M 3/00

(52) U.S. Cl. CPC C12M 23/16; C12M 21/08; C12M 25/16

(57) ABSTRACT

An open channel microfluidic device for use in tissue culture is described. The open channel microfluidic device is used as a patterning rail in a cell culture system to create suspended tissues with cells suspended in a hydrogel. The present disclosure also provides methods of preparing a tissue using the open channel microfluidic device described herein.

### CROSS-REFERENCE TO RELATED APPLICATION

This application claims priority to U.S. Provisional Patent Application No. 63/503,677, filed on May 22, 2023, which is incorporated herein by reference in its entirety as if fully set forth.

### STATEMENT REGARDING FEDERALLY SPONSORED RESEARCH OR DEVELOPMENT

This invention was made with government support under Grant No. 5F30HL158030-02, awarded by the National Heart, Lung, and Blood Institute and Grant No. 1R35GM128648-01, awarded by the National Institute of General Medical Sciences. The government has certain rights in the invention.

### BACKGROUND

Tissue engineering is a promising area for the future, hoping to solve the problems of patients suffering from irreversible organ damage caused by diseases, injuries and degeneration. Microfluidic devices can be used for culturing cells in tissue engineering. Such devices include features such as channels, chambers, and wells.

### BRIEF DESCRIPTION OF THE FIGURES

FIG. 1 illustrates an example environment in which a suspended tissue is created using a cell culture system.

FIG. 2 illustrates an example process for generating a suspended tissue.

FIG. 3 illustrates an example process for producing a patterning rail.

FIGS. 4A-4G illustrate a workflow of generating single and multi-region suspended tissues using the STOMP platform. (4A) Image of STOMP platform, including a removable patterning device containing an open channel that interfaces with the tissues posts. The patterning device is held in place to the post array with holding clips. (4B) Schematic of the STOMP platform. The hydrogel is pipetted into the open channel, where the hydrogel will flow via surface-tension drive forces across the open channel and will anchor onto the suspended posts, thus generating a free-standing suspended tissue. (4C) Side view of what the resulting suspended tissue looks like when cultured in a 24-well plate. (4D) Workflow of patterning a single region hydrogel, where the composition is the same across the tissue. (4E) Fluorescent image of 3T3 mouse fibroblast cells patterning using STOMP. The outer region of the tissues contain 3T3 cells dyed with CellTracker Green (green) and were pipetted first. The middle region contains 3T3 cells dyed with CellTracker Red (magenta). (4F) Top-down view of the capillary pinning features along the open channel that is used to pin the fluid front, and workflow of patterning suspended tissue comprising three distinct regions. Corresponding video stills show patterning of purple-colored agarose in the outer regions first, followed by patterning yellow-colored agarose in the middle region. (4G) Side view image of multi-region agarose suspended hydrogel constructs.

FIGS. 5A-5D illustrate different configurations of multi-region STOMP, including patterning a “suspended core”. By changing the positions/number of pinning features, a range of distinct regions can be patterned within a tissue. Here, patterning three regions is demonstrated (5A), two regions (5B), or a single region (5C) within a single tissue. Additionally, after removal or the first “core region” patterning device, a second “outer core” patterning device that has an open channel larger than that of the first patterning device can be added. This outer core can then be patterned around the first hydrogel, thus generating a suspended tissue that is completely encapsulated by another hydrogel; this configuration is referred to as a suspended core. (5D) Workflow of how to pattern a suspended tissue with an inner region and outer region.

FIGS. 6A-6C illustrate comparison of STOMP method with the traditional casting method when making periodontal ligament tissues (PDLs). (6A) Workflow of generating suspended tissues with the casting method. To form free-standing suspended tissues (e.g., a tissue anchored between two posts), the casting method is commonly used; in the example shown here, agarose is poured into a well plate and stamped to form an well that can hold 100 uL of a cell-laden precursor liquid hydrogel solution (such as collagen, as is the case with generating PDL tissues). A PDMS post array is then inserted into the mold with the cell-laden hydrogel precursor solution and cultured, where the cells compact and anchor onto the PDMS posts. After two days in culture (in the case with culturing cells in collagen), the PDMS post array can be removed from the mold to generate free-standing tissues that can then be cultured in a separate well plate. (6B) Workflow of generating free-standing suspended tissues using the STOMP device. The cell-laden precursor liquid hydrogel solution can be pipetted directly into the open channel of the patterning rail. The STOMP device containing the cell-laden hydrogel is then cultured for two days, relying on cell compaction of the hydrogel away from the channel walls to then allow removal of the patterning device. (6C) Image of free-standing PDL tissues (PDL cells embedded in collagen) made from the casting method and the STOMP method.

FIGS. 7A, 7B illustrate patterning a bone-ligament junction in periodontal tissue. (7A) Workflow of patterning a bone-ligament junction in periodontal tissue using STOMP. (7B) Fluorescent microscopy image of F-actin (green) and smooth muscle actin (magenta) in tissue composed of osteoblast cells in the outer region and periodontal ligament (PDL) cells in the middle region.

FIGS. 8A-8E illustrate patterned engineered heart tissues using implementations described herein. (8A) Conditions tested for modeling a fibrotic region of heart tissue using STOMP. (8B) Representative brightfield and immunofluorescent images of a control EHT seeded with HS5-GFP human bone marrow stromal cells on the two outer regions near the flexible and rigid posts, and HS5-mCherry cells in the center region. (8C) Beat Frequency. While all control and fibrotic EHTs develop effective electromechanical coupling and are able to follow the pacing frequency of 1.5 Hz (i), fibrotic EHTs show an elevated spontaneous beat rate (ii). Control n=12, Fibrotic n=14. (8D) Diastolic Function. Fibrotic EHTs show altered time to 50% relaxation (i) with no change in the time to 90% relaxation (ii). Control n=12, Fibrotic n=14. (8E) Systolic Function. Fibrotic EHTs show no differences in maximum twitch force (i), specific force (ii), or shortening velocity (iii), but do present with an altered time to peak (iv). Control n=12, Fibrotic n=14. Each shape (triangle, circle, square) represents an independent experiment. Each data point is a separate tissue.

FIGS. 9A, 9B illustrate patterning lung tissue with varying stiffness regions for modeling IPF. (9A) Schematic of workflow for patterning multi-stiffness tissue with human lung fibroblasts (LF) and image of tissue section of IPF-diseased lung pentachrome-stained to show mature fibrosis (yellow) and immature fibrosis (blue-green) (image taken from Cool et al., 2016). (9B) Stitched confocal image of patterned collagen tissue containing myofibroblasts and fibroblasts immunostained to visualize myofibroblasts (green) and nuclei (blue).

FIG. 10 illustrates utilizing STOMP to pattern engineered myotendinous junction constructs (EMTJCs). A myotendinous junction (native physiology shown in (i)) is replicated within (ii) STOMP-patterned engineered tissue utilizing primary tenocytes and an immortalized murine myoblast cell line.

FIGS. 11A-11E illustrate patterning skeletal muscle (SkM) tissue with a healthy and diseased region for modeling facioscapulohumeral dystrophy (FSHD). (11A) Schematic of patterning SkM tissue with diseased (MB200) cells in the middle and healthy (MB135) cells in the outer regions. (11B) Maximum z projection confocal image of resulting patterned tissue after 7 days of differentiation. MB135 cells were dyed with CellTracker Red and MB200 cells were dyed with CellTracker Green prior to patterning. (11C) RT-qPCR gene expression of differentiation genes i) MYH2, ii) CKM, and iii) DES in healthy (MB135) and FSHD diseased (MB200) differentiation myotubes. All values were normalized to the reference gene RPL27; relative mRNA levels for each gene were normalized to that in 2D differentiated healthy myotubes. (11D) RT-qPCR gene expression of i) DUX4 and ii) ZSCAN4 in FSHD diseased (MB200) myotubes. All values were normalized to the reference gene RPL27; relative mRNA levels for each gene were normalized to that in 2D differentiated diseased myotubes. (11E) Immunofluorescent staining of 3D MB200 tissue cryosections after 7 days differentiation.

FIGS. 12A-12E illustrate degradable inner channel for patterning tissues and materials that do not compact. (12A) Isometric view of STOMP device with the outer channel. (12B) Front (y-z plane) cross section and (12C) side (x-z plane) cross section. (12D) Demonstration of patterning with colored agarose. Blue-colored agarose is pipetted first to fill the inner channel and the green-colored agarose is pipetted second to generate the suspended tissue. (12E) Image of fully removed PEG tissue patterned using device.

### DETAILED DESCRIPTION

Various implementations of the present disclosure relate to an open channel microfluidic device for use in tissue culture. The open channel microfluidic device is used as a patterning rail in a cell culture system to create suspended tissues. A benefit of suspended tissue culture is that cells can experience and respond to mechanical forces that are not achieved in other culture systems.

In particular implementations, the open channel microfluidic device can be used for tissue engineering of any tissue type that includes cells that can modify their extracellular matrix. In particular implementations, the open channel microfluidic device can be used for tissue engineering of heart tissue. In particular implementations, the open channel microfluidic device can be used for tissue engineering of a periodontal tissue and of a muscle tissue. In particular implementations, the open channel microfluidic device can be used for tissue engineering of a tissue interface between two different tissues. For example, the open channel microfluidic device can be used for tissue engineering of a muscle-tendon tissue. In particular implementations, the open channel microfluidic device can be used for tissue engineering of artificial meat. In particular implementations, the open channel microfluidic device can be used for tissue engineering of artificial meat for consumption.

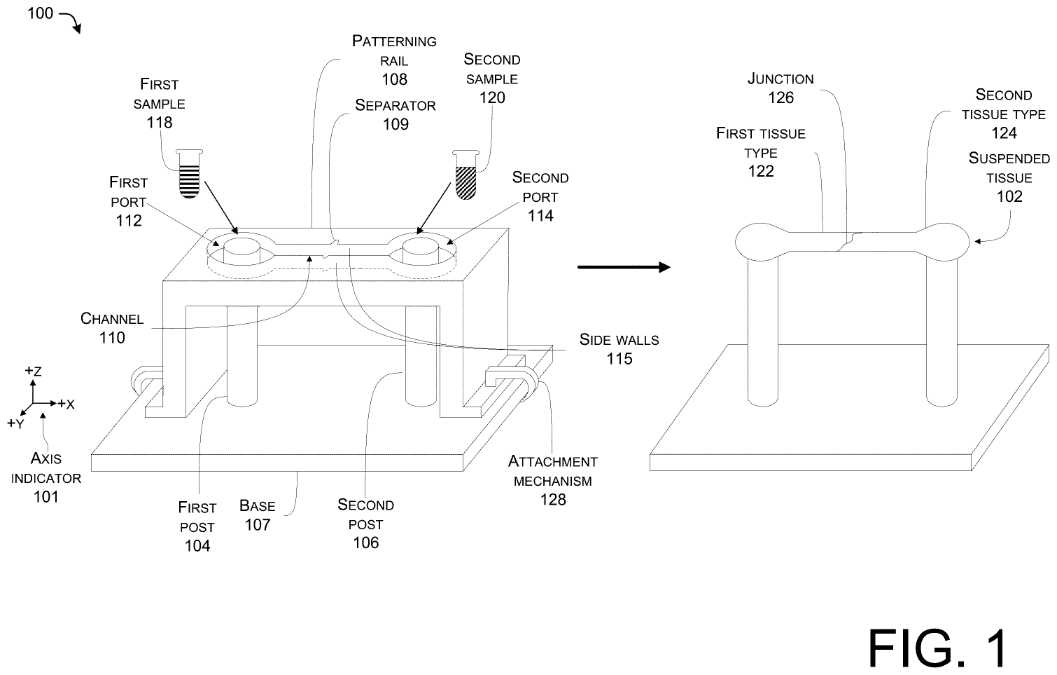

FIG. 1 illustrates an example environment 100 in which a suspended tissue 102 is created using a cell culture system. The suspended tissue 102 may include one or more types of cells or from a living organism. For example, the suspended tissue 102 may include fibroblasts, epithelial cells, alpha cells, beta cells, delta cells, cardiomyocytes, endothelial cells, hepatocytes, hepatic stellate cells, Kupffer cells (KCs), liver sinusoidal endothelial cells (LSECs), endocrine cells, myoblasts, chondrocytes, neurons, osteoblasts, osteoclasts, kidney cells, stromal cells, stem cells, induced pluripotent stem cells, embryonic stem cells, mesenchymal stem cells, hematopoietic stem cells, tenocytes, or myocytes. The suspended tissue 102 may include human cells, non-human primate cells, mammalian cells, murine cells, rat cells, or cells from another animal. In some examples, the suspended tissue 102 includes one or more types of modified cells, engineered cells, synthetic cells, chimeric cells, or the like. The suspended tissue 102, in various cases, includes a hydrogel. The hydrogel may include at least one of gelatin, fibrin, collagen, chitosan, fibronectin, laminin, alginate, Matrigel, hyaluronic acid, polyethylene glycol (PEG), or polyacrylamide. The suspended tissue 102, in various implementations, may be disposed on a first post 104 and a second post 106. A post array may include a base 107 that is connected to the first post 104 and the second post 106. FIG. 1 includes an axis indicator 101, wherein height is measured in the direction of the arrow of the z-axis, and the “top” of an object described herein refers to the part of an object with the greatest height.

The first post 104 and the second post 106, in some examples, include glass, plastic, or metal. In some examples, the first post 104 and the second post 106 include a rigid material and/or a flexible material. The first post 104 and the second post 106 may include, for instance, borosilicate glass, soda-lime glass, fused silica, silicone, polydimethylsiloxane (PDMS), polyurethane (PU), thermoplastic elastomers, fluoropolymers, polyvinyl chloride (PVC), polystyrene (PS), polycarbonate (PC), polyethylene (PE), polypropylene (PP), cyclic olefin (CO) polymer, CO copolymer, acrylonitrile butadiene styrene (ABS), poly(methyl methacrylate) (PMMA), stainless steel, stainless steel, titanium, a metal alloy, or the like. In particular implementations, the first post 104 and/or the second post 106 includes a flexible material and a glass rod. In some examples, the first post 104 includes PDMS and a glass rod, and the second post 106 includes PDMS. In some examples, the first post 104 includes silicone and a glass rod, and the second post 106 includes a silicone. In various instances, the first post 104 and/or the second post 106 are configured to bend at a radius of curvature in a range of 0.1 to 24 inches. In various cases, the first post 104 and/or the second post 106 are configured to bend at a radius of curvature in a range of 1 to 12 inches. In some examples, the first post 104 and/or the second post 106 may be a height in a range of 4 to 20 millimeters (mm). For instance, the first post 104 and the second post 106 may have a height of 12 mm. In various cases, the first post 104 and the second post 106 may have a diameter in a range of 0.1 to 5 millimeters (mm). For example, the first post 104 and the second post 106 may have a diameter of 1.5 mm. In some cases, the first post 104 and/or the second post 106 may have a T-shaped cap on the top of the post. For instance, the first post 104 and the second post 106 may have a T-shaped cap with a diameter in a range of 0.1 to 4 mm and a thickness of 0.1 to 4 mm. For example, the T-shaped cap may have a diameter of 2 mm and a thickness of 0.5 mm. In some examples, the first post 104 or the second post 106 include a magnetic material. The magnetic material may include a rare earth magnet, a ferromagnetic material, an electromagnet, or the like. For instance, a rare earth magnet may be disposed on, around, or within the second post 106.

In various examples, it may be beneficial to improve control over the geometry, structure, and spatial distribution of the suspended tissue 102. Currently, suspended tissues are generally produced using a casting method. However, a casting method provides limited control over the geometry, structure, and spatial composition of a suspended tissue. For instance, spatially separating different cell types in the suspended tissue 102 may better model a disease state or interactions between different cell types. In some examples, generating the suspended tissue 102 that includes two or more cell types can enable a more comprehensive understanding of the mechanical properties or cellular signaling at a tissue junction. In some cases, it may be beneficial to generate a thin suspended tissue if resources (e.g., cells, cell culture reagents, etc.) are limited.

These issues can be addressed by using a patterning rail 108 to generate the suspended tissue 102. In various implementations, the patterning rail 108 can be removably attached to the first post 104 and the second post 106. In some examples, at least one patterning rails 108 can be removably attached to the post array. A channel 110 that is configured to hold the suspended tissue 102 is disposed, in some examples, in the patterning rail 108. In various implementations, the channel 110 is configured to spontaneously propel, along a length of the channel 110 in the x-direction, a fluid sample that includes a cell composition. That is, the dimensions and material composition of the channel 110 causes the fluid sample to spontaneously flow through the channel 110 via capillary action. The patterning rail 108, in various implementations, is configured to be inserted into a cell culture vessel to culture the cell composition. In some implementations, the patterning rail 108 may include a separator 109 configured to spatially separate two or more different cell compositions. Accordingly, the patterning rail 108 can be used to generate the suspended tissue 102 that includes two or more tissues that are spatially separated.

The patterning rail 108, in some examples, includes glass, plastic, or metal. Examples of glass include borosilicate glass, soda-lime glass, fused silica, or the like. Examples of plastic include silicone, PDMS, PU, thermoplastic elastomers, fluoropolymers, PVC, PS, PC, PE, PP, CO polymers, CO copolymer, ABS, PMMA, or the like. Examples of metal include stainless steel, titanium, or any other suitable metal. In some examples, the patterning rail 108 includes a biocompatible polymer, such as polyethylene glycol (PEG), poly(lactic-co-glycolic acid) (PLGA), poly(lactic acid) (PLA), polyhydroxyalkanoates (PHA), chitosan, gelatin, or the like. In some examples, a surface of the patterning rail is coated. For example, the patterning rail 108 may be coated with a blocking agent configured to prevent attachment of the suspended tissue 102 to the patterning rail 108. Examples of blocking agents include bovine serum albumin (BSA), casein, milk, PEG, or the like.

In various implementations, the patterning rail 108 includes the channel 110 that connects a first port 112 and a second port 114. In some examples, the channel 110 includes two side walls 115 surround a region. The region is configured to hold the suspended tissue 102. In various examples, the two side walls 115 are parallel to each other and are separated by a space. The channel 110 is configured spontaneously wick a fluid along the channel 110 in the x-direction. In some examples, the dimensions of the region are determined using the following equation:

$$\frac{w}{h} < cos θ$$

where w is the width of the region in the y-direction (i.e., the distance between the two side walls), h is the height of the region in the z-direction (i.e., the height of the two side walls), and Θ is the contact angle of a sample to each of the two side walls. The channel 110 may include more than one region, and each of the regions may have the same or different dimensions.

The first port 112 and the second port 114 are configured to receive a fluid sample and introduce the fluid sample to the channel 110. For instance, the first port 112 may receive a first sample 118, and the second port may receive a second sample 120. The first sample 118 and the second sample 120 may flow into the channel 110. The first post 104 and the second post 106 are, in various cases, disposed within the first port 112 and the second port 114, respectively. The first port 112 and the second port 114 are connected to the channel 110. The two side walls of the channel 110, in some examples, connect between the first port 112 and the second port 114. The dimensions of the channel 110, the first port 112, and the second port 114, in various examples, can depend on the principles of capillary action.

In some implementations, the patterning rail 108 is configured to cause spontaneous flow of the first sample 118 and the second sample 120 without an external force. In some implementations, a flow of the first sample 118 and/or the second sample 120 may be controlled by an external device. For instance, the external device may include a pump, such as a centrifugal pump, a positive displacement pump, a diaphragm pump, an electromechanical pump, or a gear pump, or any other device known in the art.

In some examples, a separator 109 is configured to separate two cell types in the suspended tissue 102. For instance, the separator 109 may be configured to control a capillary flow of the first sample 118 from the first port 112 to the separator 109 and a capillary flow of the second sample 120 from the second port 114 to the separator 109. Accordingly, the separator 109 may enable two or more tissue types to be spatially separated in the suspended tissue 102. In some examples, the separator 109 may include a capillary pinning ridge configured to prevent capillary flow and enable formation of an interface between the first sample 118 and the second sample 120. In some examples, the separator 109 may be a removable component or a component that degrades or dissolves over time. For instance, the separator 109 may include a biodegradable polymer (e.g., polylactic acid (PLA), polyglycolic acid (PGA), polycaprolactone (PCL), or the like) or a bioresorbable material (e.g., PLA, PGA, PCL, collagen, hyaluronic acid, or the like).

In some implementations, a third sample can be applied to the channel 110. For instance, the first sample 118 may be applied to the first port 112, the second sample 120 may be applied to the second port 114, and the third sample may be applied to the channel 110. In some examples, the channel 110 includes two or more separators. The third sample may be applied between the two separators to spatially separate the third sample from the first sample 118 and the second sample 120.

The first sample 118 and the second sample 120 may include different cell types, experimental conditions, ECM components, or the like. In various examples, the first sample 118 and the second sample 120 includes growth factors, such as epidermal growth factor (EGF), fibroflast growth factor (FGF), vascular endothelial growth factor (VEGF), transforming growth factor-beta (TGF-β), insulin-like growth factor (IGF), or any other growth factor. The first sample 118 and the second sample 120, in some examples, are configured to generate (e.g., mature into) a first tissue type 122 and a second tissue type 124. In some examples, the suspended tissue 102 includes 2, 3, 4, 5, 6, or more than 6 different tissue types. Each of the multiple tissue types may be separated by a junction 126. In some examples, the suspended tissue 102 may include three tissue types, wherein the first tissue type 122 is in contact with a third tissue type, the third cell type is in contact with the first tissue type 122 and the second tissue type 124, and the second tissue type 124 is in contact with the third tissue type. In some examples, the suspended tissue 102 may include two or more tissue types that are not spatially separated (e.g., the cells of each tissue type are interspersed). In various cases, the suspended tissue 102 may include heart tissue, periodontal tissue, muscular tissue, lung tissue, liver tissue, kidney tissue, epithelial tissue, connective tissue, adipose tissue, or any other tissue type. In some examples, the suspended tissue 102 includes engineered cells. For instance, the suspended tissue 102 may include artificial meat. The suspended tissue 102, in some examples, includes a hydrogel with a thickness in range of 100 μm to 10 mm. In some examples, the thickness of the hydrogel is in a range of 1 to 7 mm.

In some implementations, an attachment mechanism 128 is configured to removably secure the patterning rail 108 to the post array. For instance, the patterning rail 108 may be coupled to at least one of the first post 104, the second post 106, or the base 107. The attachment mechanism 128 may include a clip, a clamp, a latch, or the like. For example, the attachment mechanism 128 may include a first clip and a second clip may be configured to secure the patterning rail 108 to the base 107. In some examples, the first clip and the second clip are configured to secure the patterning rail 108 to the first post 104 and the second post 106, respectively. In some examples, the patterning rail 108 includes the attachment mechanism 128, and the attachment mechanism 128 is configured to couple to the first post 104 and the second post 106. For example, the attachment mechanism 128 may include an interlocking joint (e.g., a notch, a groove, a slot, etc.), a snap fit connection (e.g., a ridge, a tab, etc.), a clamp, a latch, or the like.

Based on securing the patterning rail 108 to the post array, the patterning rail 108 may be inserted into a cell culture vessel. The cell culture vessel may include one or more wells, and a cell culture media may be disposed in the one or more wells. A size of each of the one or more wells may be larger than a size of the patterning rail. A height of each of the one or more wells may be greater than a height of the patterning rail attached to the post array measured in the z-direction. The cell culture media may include Dulbecco's Modified Eagle's Medium (DMEM), RPMI 1640 Medium, Minimum Essential Medium (MEM), F-12 Nutrient Mixture, or any other cell culture media. The cell culture media may be selected based on the first sample 118 or the second sample 120. For instance, the cell culture vessel may be a well plate.

In some implementations, the patterning rail 108 includes a support configured to separate the suspended tissue 102 from the patterning rail 108. The support, in various cases, includes a biocompatible polymer, such as PEG, PLGA, PLA, PHA, chitosan, gelatin, or the like. In some examples, the support is generated using a crosslinker, such as PEG diacrylate (PEGDA), PEG dimethacrylate (PEGDMA), N,N′-methylenebis(acrylamide) (BIS), PEG divinyl sulfone (PEGDVS), or the like. The support may be configured to be degraded, such as by an enzyme, photodegradation, chemical degradation, or another suitable method. For instance, the support may be configured to be degraded by sortase. In some examples, the patterning rail 108 is generated using a first crosslinker, and the first crosslinker is different than a second crosslinker used to generate the support.

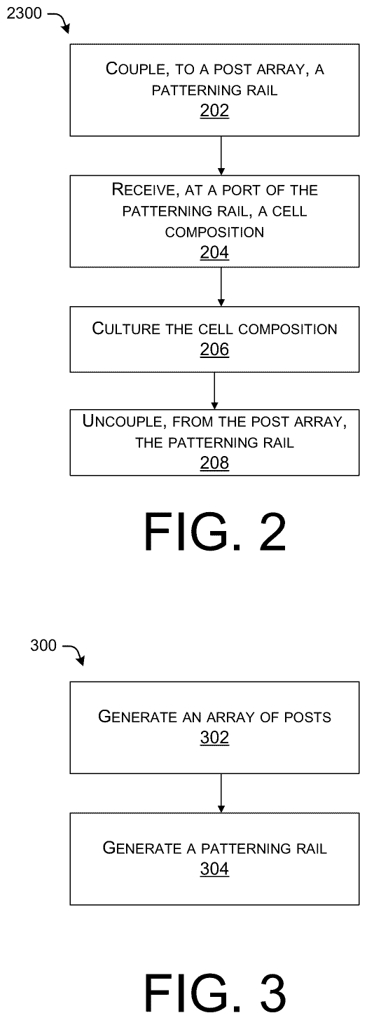

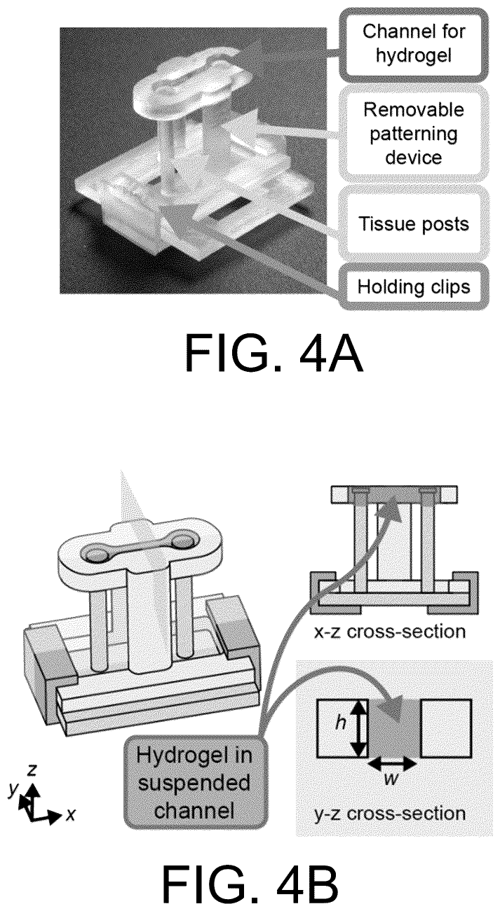

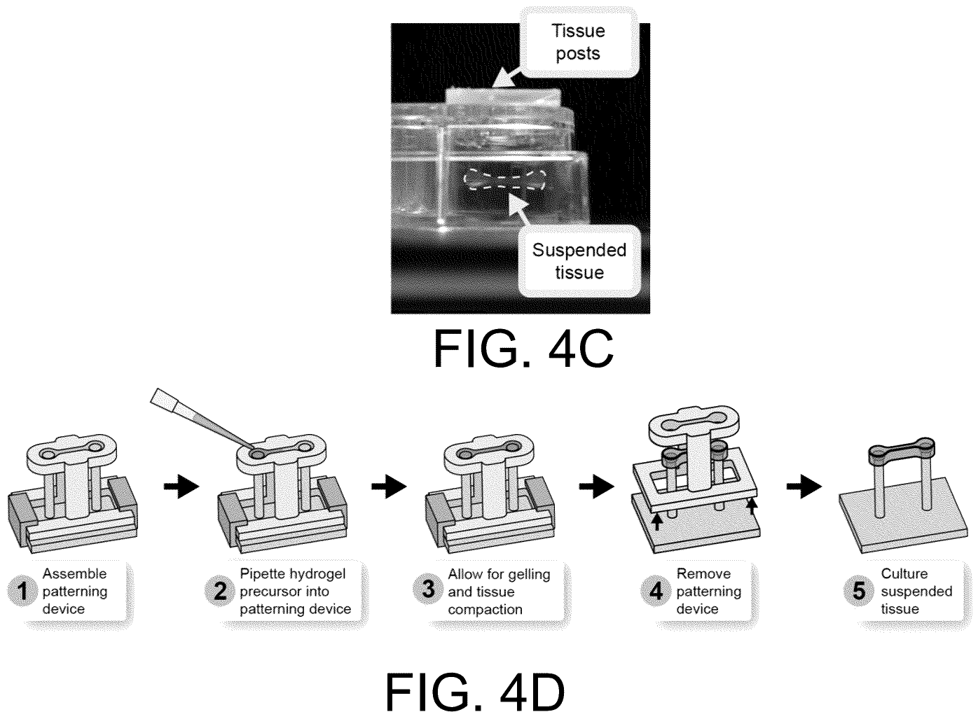

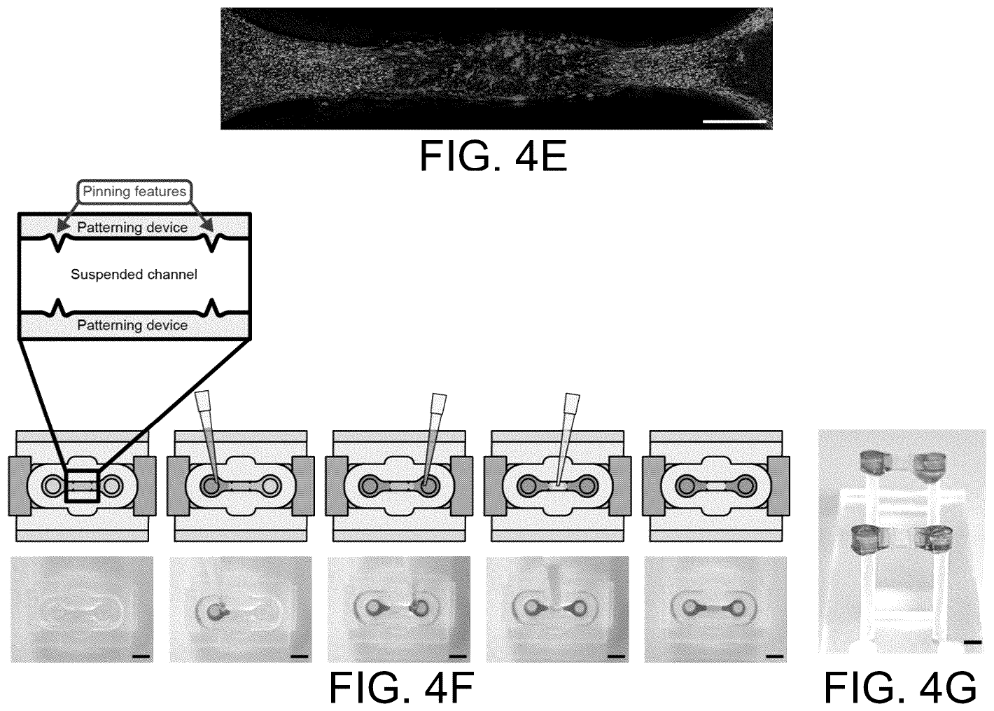

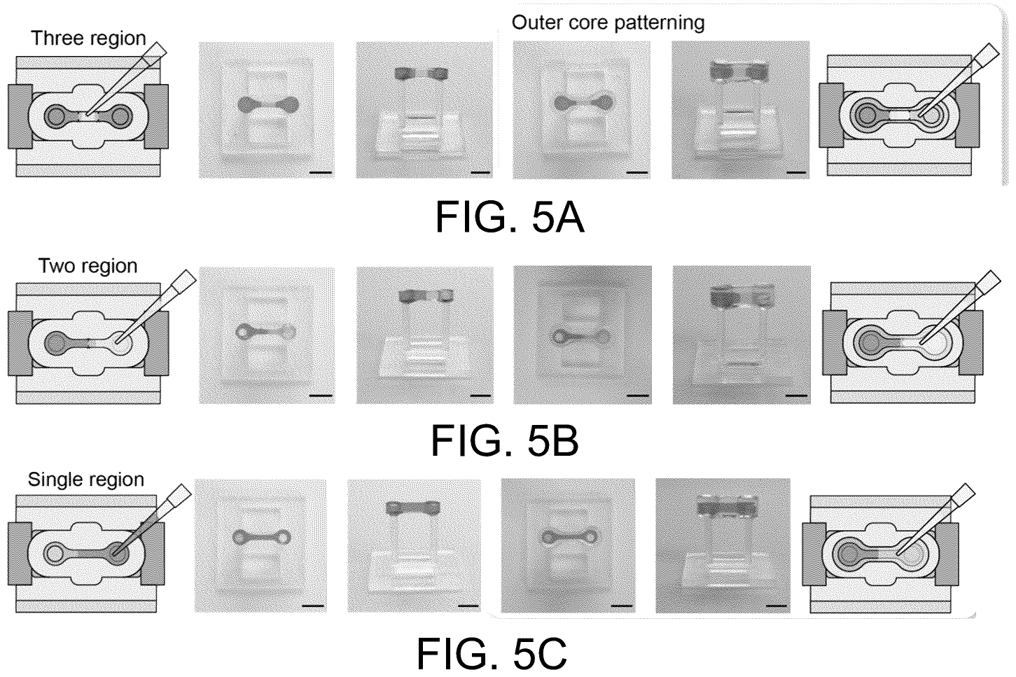

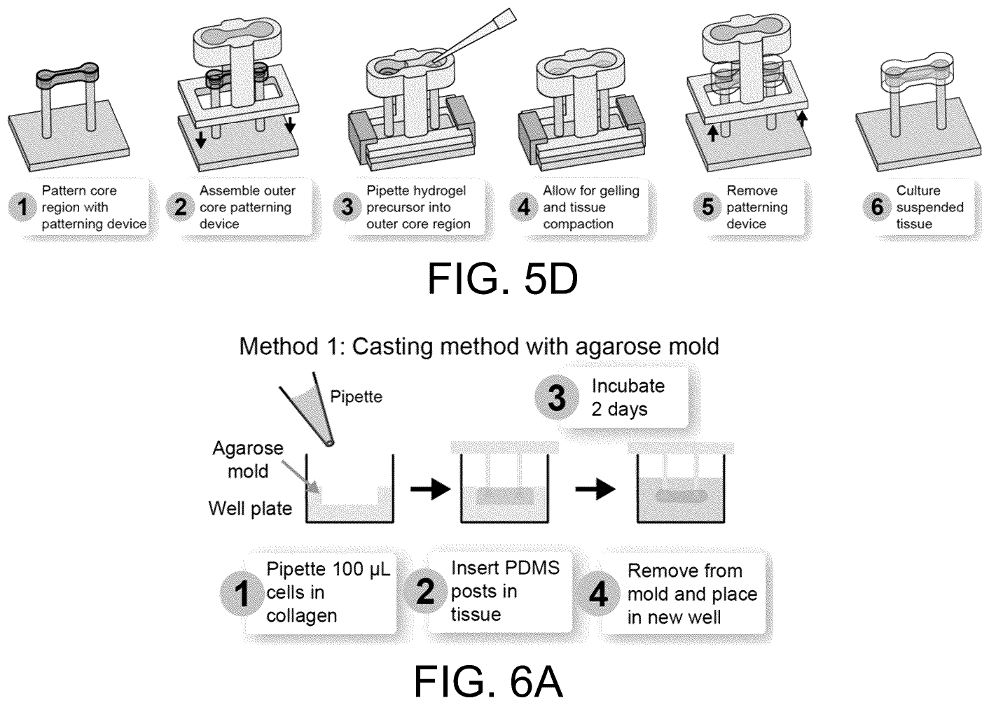

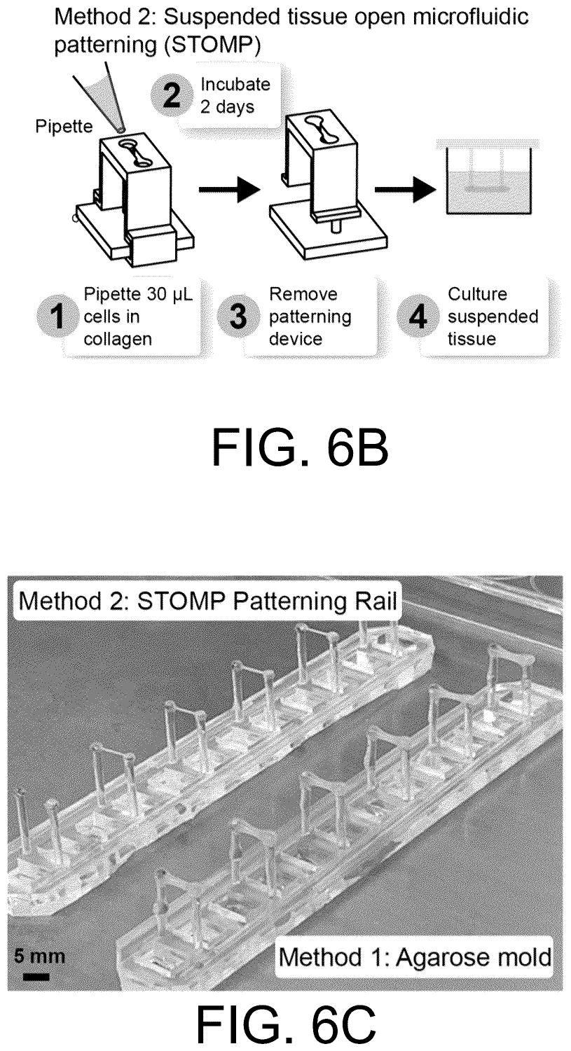

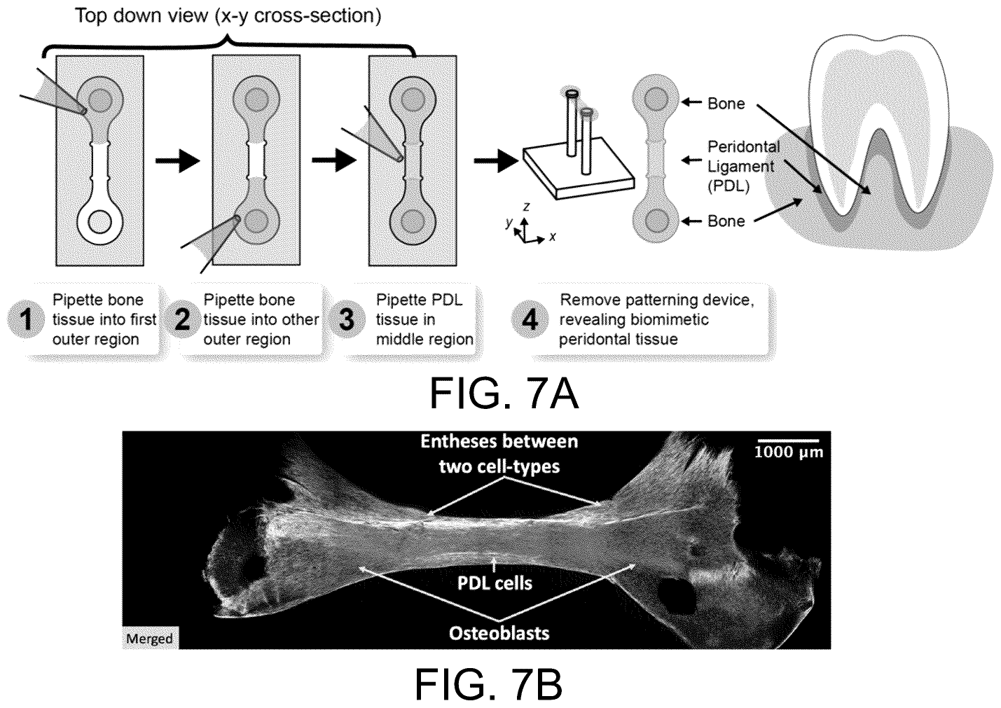

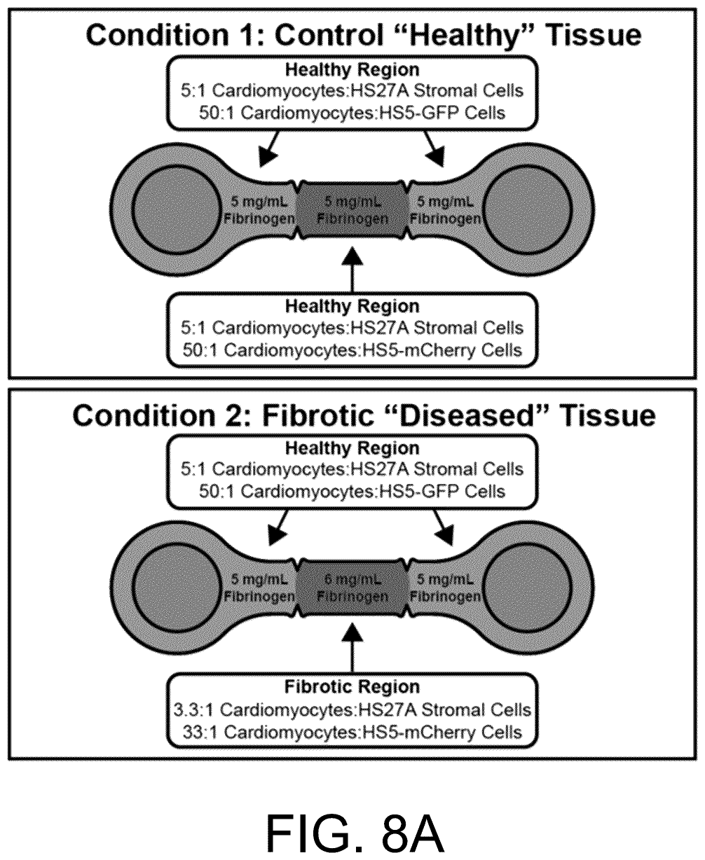

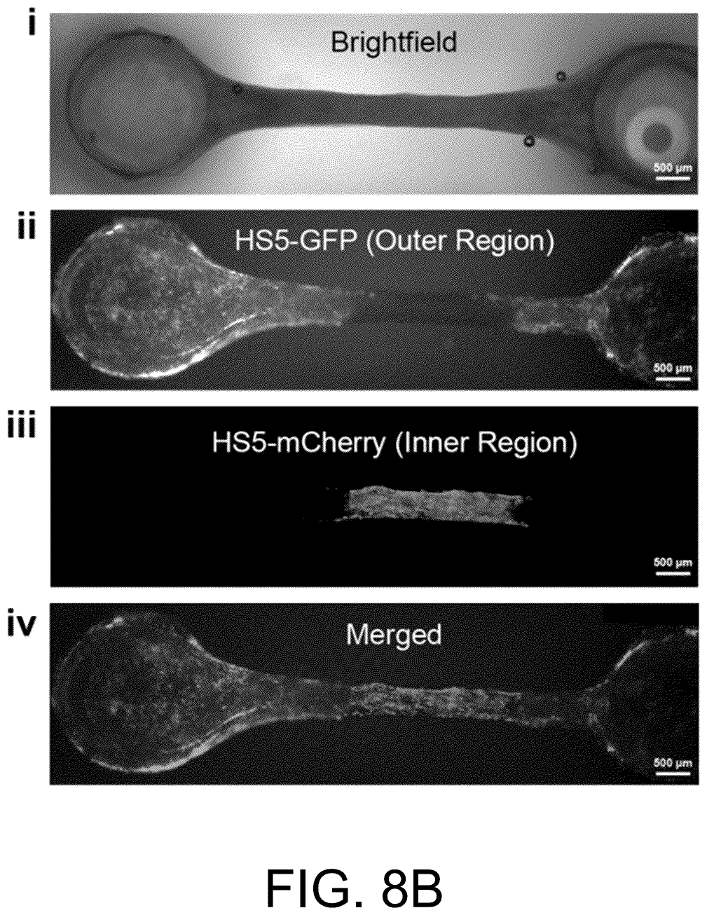

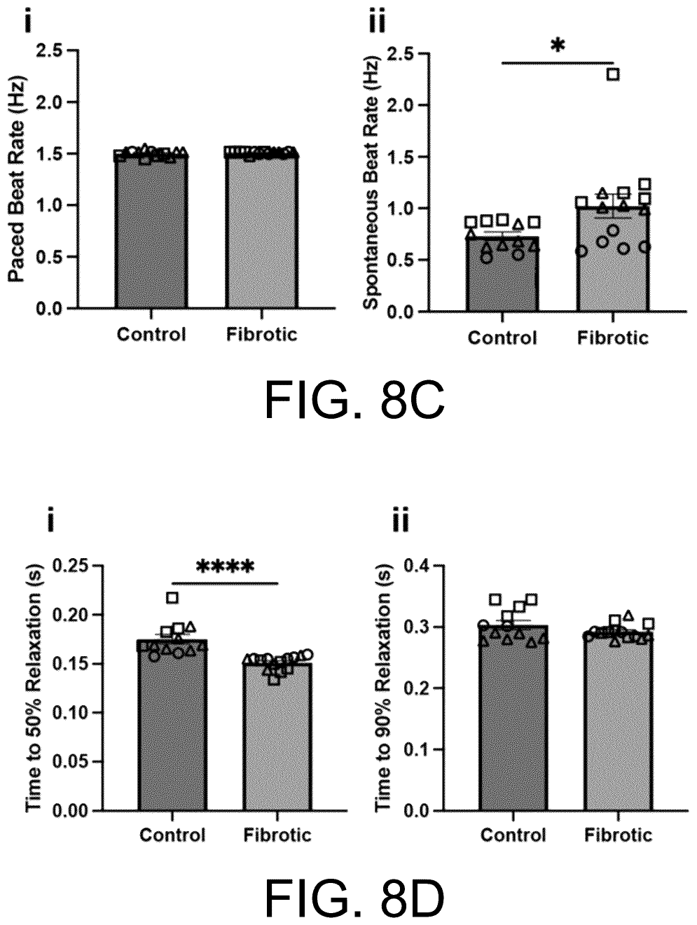

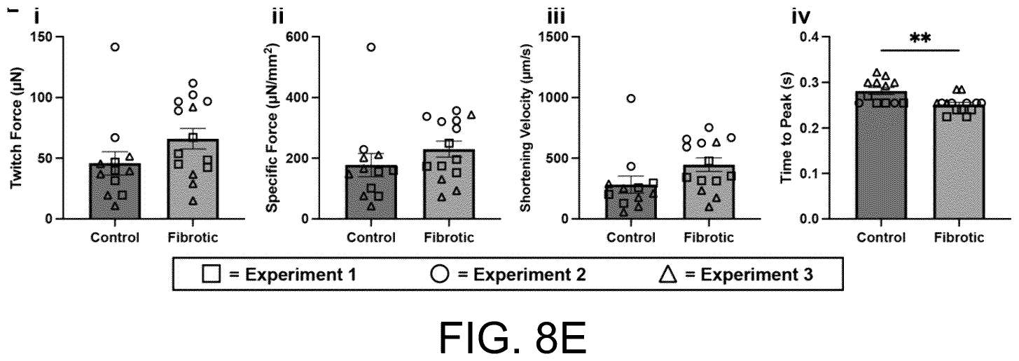

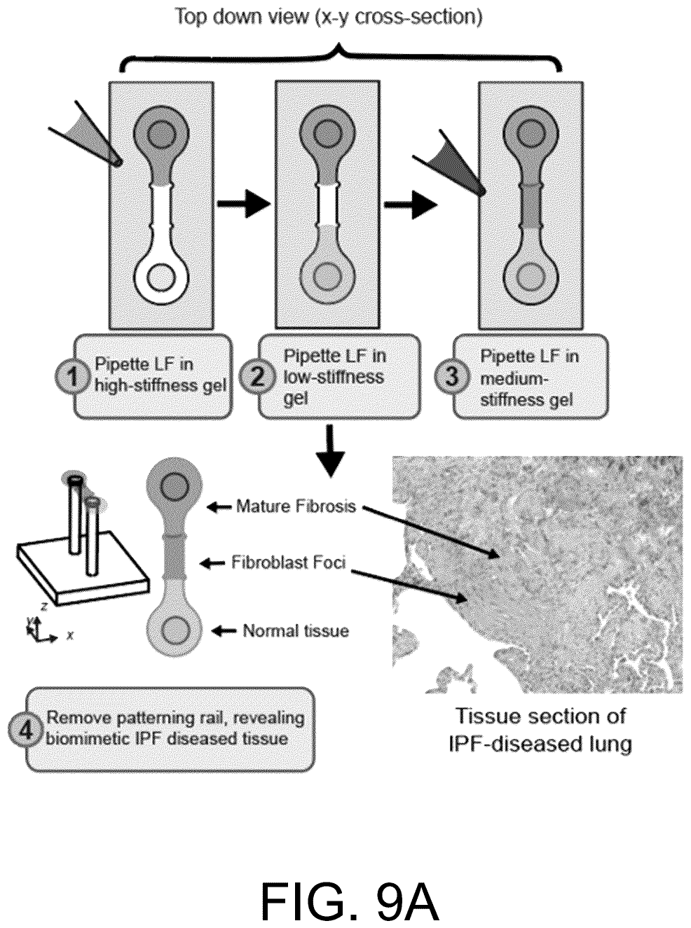

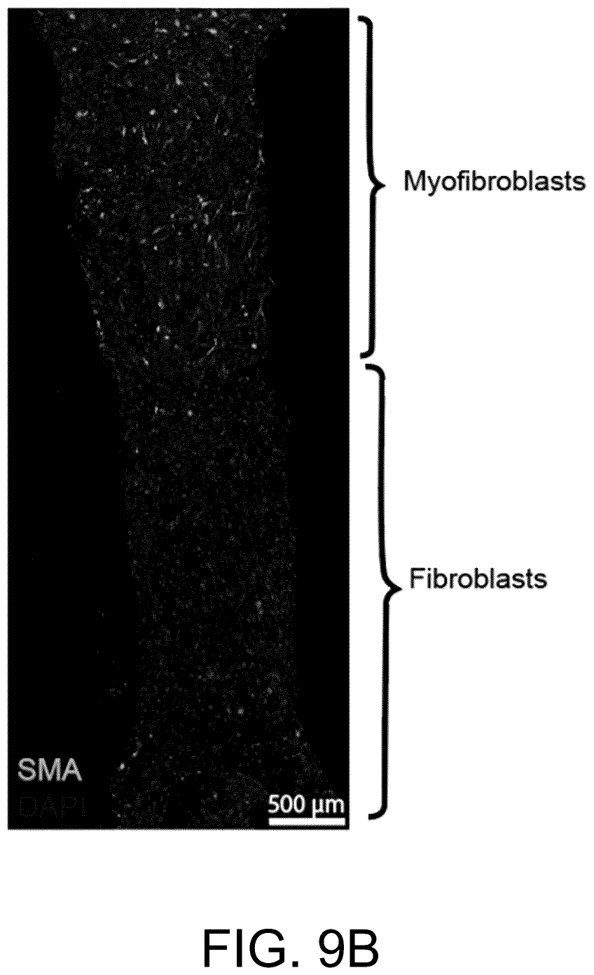

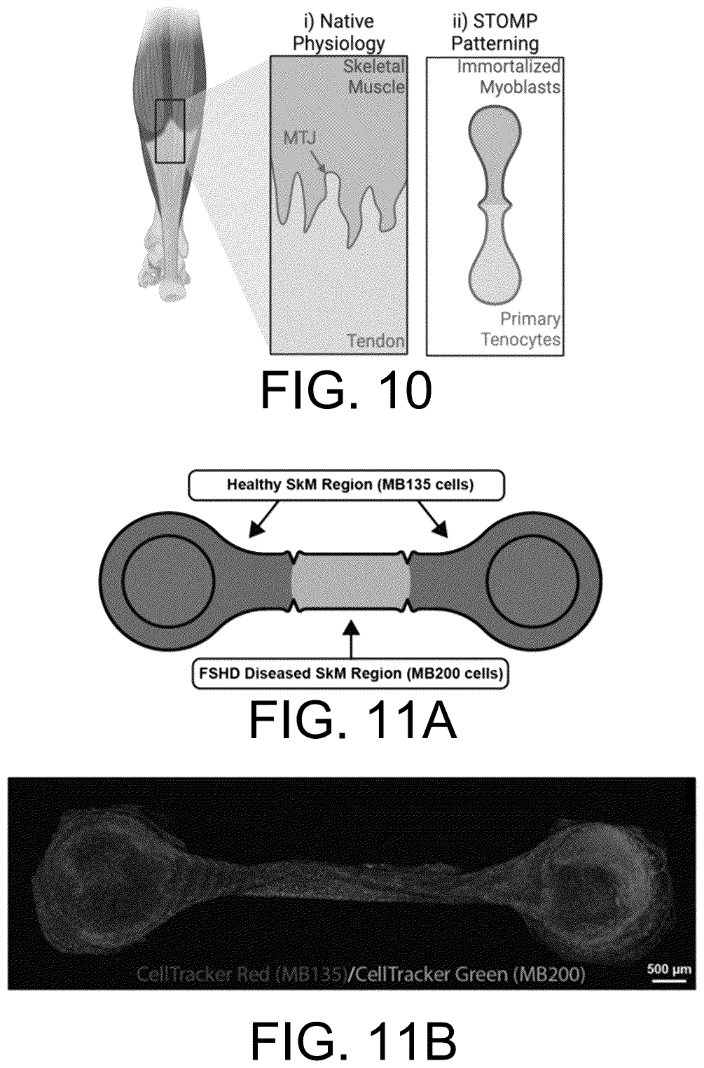

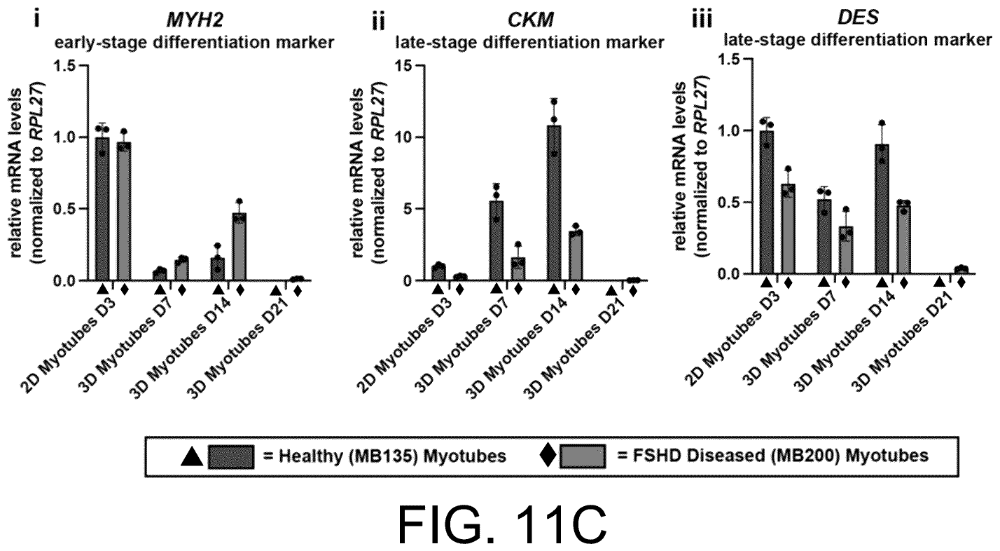

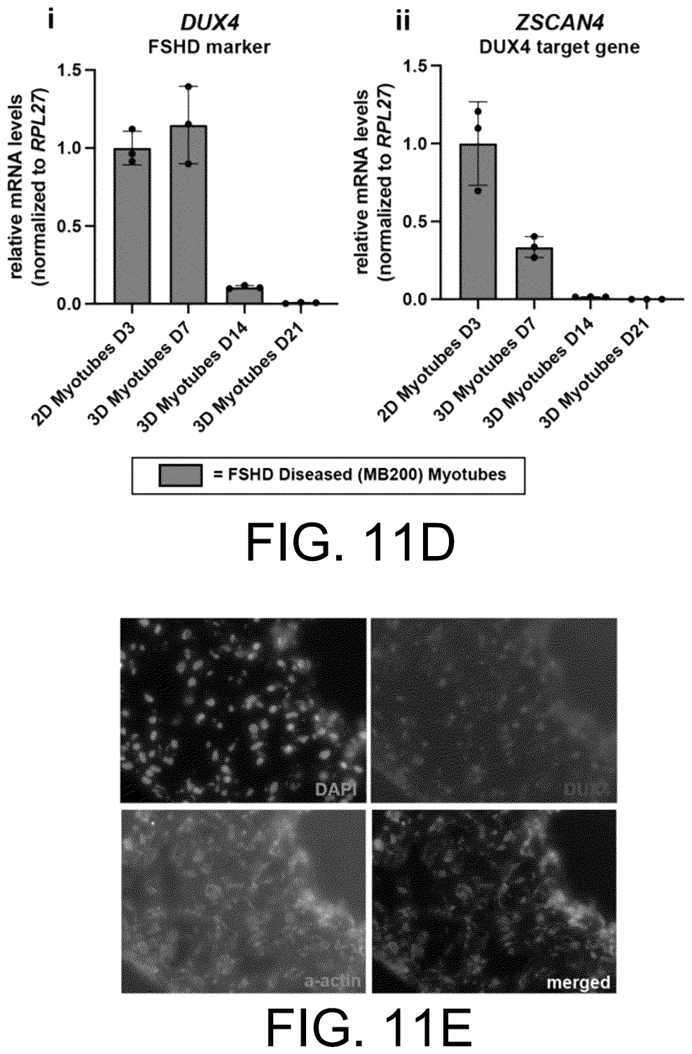

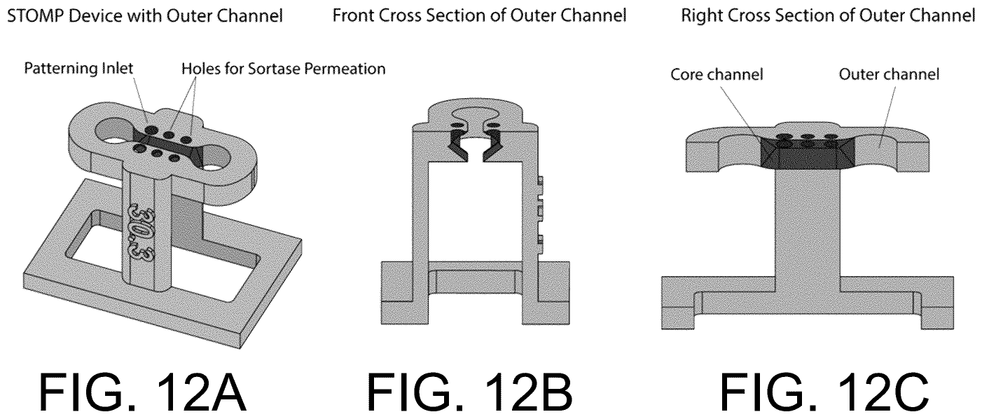

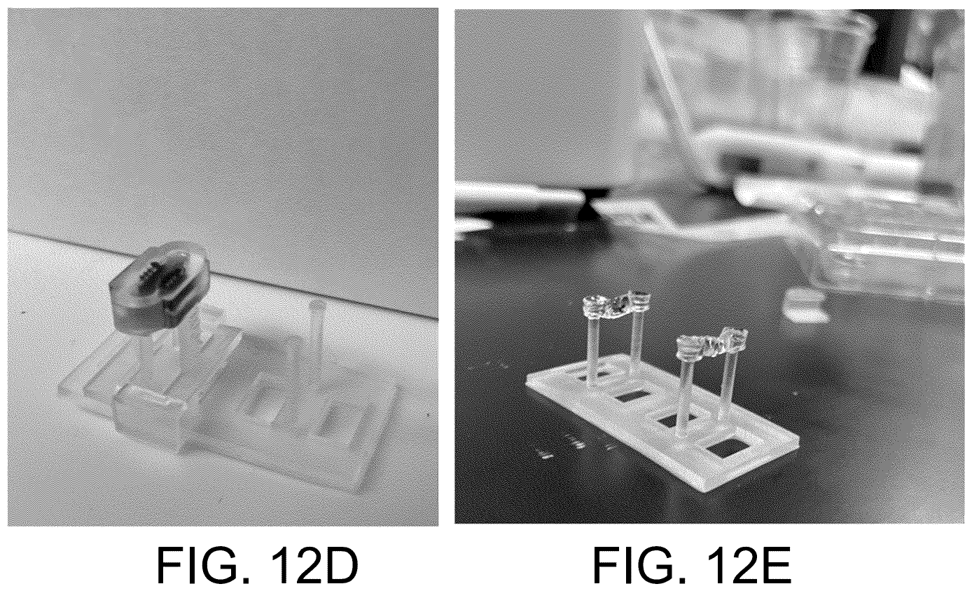

FIG. 2 illustrates an example process 200 for generating a suspended tissue (e.g., the suspended tissue 102). The process 200 may be performed by a device (e.g., a fluidic device, a microfluidic device, a robotic device, or the like) or an entity (e.g., a technician, a researcher, a trained user, or the like). In some examples, one or more steps of process 200 may be omitted.

At 202, the patterning rail (e.g., the patterning rail 108) is coupled to a post array that includes a first post (e.g, the first post 104) and a second post (e.g., the second post 106). The first post, in various cases, is disposed within a first port (e.g., the first port 112) of the patterning rail, and the second post is disposed within a second port (e.g., the second port 114) of the patterning rail. In some examples, the first post and the second post are part of an array of posts that is configured to couple to multiple patterning rails. In some examples, the array of posts may be disposed on or within a well plate (e.g., a 6-well plate, a 24 well plate, a 48-well plate, a 96-well plate, or the like).

In some examples, an attachment mechanism (e.g., the attachment mechanism 128) is engaged to removably secure the patterning rail to at least one of the first post, the second post, or a base (e.g., the base 107) connected to the first post and the second post. For instance, a first clip and a second clip may attach the patterning rail to the base to prevent movement of the patterning rail relative to the base.

At 204, a first cell composition (e.g., the first sample 118) is received at the first port of the patterning rail. In some examples, an entity may pipet the first cell composition into the port. In various implementations, a second cell composition (e.g., the second sample 120) is received at the second port of the patterning rail. Based on receiving the first cell composition, the first cell composition may propel (e.g., spontaneously flow via capillary action) from the first port to a channel (e.g., the channel 110) of the patterning rail. In some examples, the first cell composition may spontaneously flow to the channel via capillary action. In some examples, the first cell composition may be propelled by an external device. Based on receiving the second cell composition, in some implementations, the second cell composition may wick from the second port to the channel. The first cell composition and the second cell composition, in various examples, may wick from the first port and the second port, respectively, to a separator (e.g., the separator 109). The separator may be configured to prevent the flow of the first cell composition and the second cell composition. In some examples, an additional volume of the first cell composition and/or an additional amount of the second cell composition may be received at the first port or the second port, respectively. Based on receiving the additional volume of the first cell composition and/or the additional amount of the second cell composition, the first sample may cell composition the second cell composition at the separator.

In some implementations, based on receiving the first cell composition, the patterning rail may be immersed in a gelling solution. The gelling solution may be configured to cause a hydrogel in the first cell composition to form a three-dimensional structure. In various examples, the gelling solution includes phosphate buffered saline (PBS), water, tris-buffered saline (TBS), HEPES buffer, Ringer's solution, or another solution. In some examples, the hydrogel may form a three-dimensional structure based on applying chemical or physical crosslinking, such as by using temperature-induced gelling, ionic crosslinking, hydrogen bonding, a crosslinking agent, UV-induced crosslinking, or the like.

At 206, the first cell composition is cultured. In various examples, a cell culture vessel may be configured to receive the patterning rail attached to the post array. The patterning rail attached to the post array may be turned 180° in the z-direction and inserted into the cell culture vessel. In various examples, the cell culture vessel includes cell culture media. The volume of the cell culture media, for instance, may enable the patterning rail to be immersed in the cell culture media when the post array is inserted into the cell culture vessel. The cell composition may be cultured in an incubator (e.g., a CO₂ incubator), a bioreactor, or the like. In some cases, the cell composition is cultured at a temperature in a range of 35 to 40° C. In some cases, the cell composition is cultured at a temperature of 37° C. The cell composition, in various examples, is cultured in an environment with a CO₂ level if a range of 4 to 10%. In various examples, the CO₂ level is in a range of 5 to 7%.

At 208, the patterning rail is uncoupled from the post array to generate a suspended tissue (e.g., the suspended tissue 102). In various examples, the suspended tissue is isolated by detaching the patterning rail from the first post and the second post, for instance, using the attachment mechanism. For example, the first clip and the second clip of the attachment mechanism may be detached from the base, and the patterning rail may be removed. In various examples, the patterning rail is removed when the suspended tissue exhibits a certain characteristic. For instance, the suspended tissue may detach from side walls (e.g., the side walls 115) of the channel, indicating that the suspended tissue has been produced. In some examples, the patterning rail is removed after a support (e.g., the support described in reference to FIG. 1) is degraded. For instance, an enzyme, a chemical, or a light may be applied to the patterning rail to specifically degrade the support. Based on degrading the support, the suspended tissue is isolated, and the patterning rail can be removed.

In some examples, the steps of process 200 may be repeated to form layers of tissue within the suspended tissue. For instance, each of the layers may include different cell types. In various implementations, the suspended tissue may be encapsulated with an engineered tissue (e.g., a synthetic tissue). In some examples, the suspended tissue may be degradable. For instance, the suspended tissue may be enzyme-degradable, enabling formation of a cavity within the engineered tissue. The engineered tissue may be used, for example, to model vascularization.

FIG. 3 illustrates an example process 300 for producing a patterning rail (e.g., the patterning rail 108) that is configured generate a suspended tissue (e.g., the suspended tissue 102). The process 300 may be performed by a device (e.g., a printer, a robotic device, or the like) or an entity (e.g., a technician, a researcher, a trained user, or the like). In some implementations, one or more of the steps of process 300 may be omitted.

At 302, an array of multiple posts (e.g., the first post 104, the second post 106, etc.) is generated. The array may be generated using casting, 3D printing, molding, machining, forming, cutting, fusing, or another suitable method. The array may include a base (e.g., the base 107) that is connected to the multiple posts. In various examples, the array includes pairs of posts, where each pair of posts includes two posts separated by a predetermined distance. In some examples, one post of a pair of posts is flexible, and the other post of the pair is rigid. The rigid post may be generated by attaching, to or within a flexible material, a rigid rod. The rigid rod may be a glass rod, a metal rod, a plastic rod, or another rod. In some examples, one or more of the multiple posts includes a magnetic material.

At 304, the patterning rail is generated. The patterning rail may be generated using 3D printing, casting, molding, machining, forming, cutting, fusing, or another suitable method. In some examples, the patterning rail includes a polymer that is generated by crosslinking, such as chemical crosslinking, enzymatic crosslinking, physical crosslinking, or the like. For instance, the patterning rail, in various cases, includes a support (e.g., the support described in reference to FIG. 1). The support may include a polymer that is generated by crosslinking. In some implementations, the patterning rail is coated. For instance, the patterning rail may be coated with a blocking agent. The patterning rail may be coated by immersion, spraying, brushing, vapor deposition, or the like.

The Exemplary Implementations and Example below are included to demonstrate particular implementations of the disclosure. Those of ordinary skill in the art should recognize in light of the present disclosure that many changes can be made to the specific implementations disclosed herein and still obtain a like or similar result without departing from the spirit and scope of the disclosure.

### Example Clauses

1. A cell culture system including:
   a patterning rail including a first port and a second port connected by an open microfluidic channel;
   an array including a first post and a second post, the first post being configured to couple to the first port and the second post being configured to couple to the second port; and
   an attachment mechanism configured to removably attach the patterning rail to the array; and
   a cell culture vessel configured to receive the array.
2. The cell culture system of clause 1, wherein the patterning rail includes a polymer, glass, plastic, or metal, and wherein the first post and/or the second post include glass, plastic, metal, silicone, flexible silicone, polydimethylsiloxane (PDMS), or a glass rod.
3. The cell culture system of clause 1 or 2, wherein the open microfluidic channel includes: walls separated by a space; and one or more regions, wherein each of the one or more regions includes a volume within the open microfluidic channel such that fluid within each region is separated.
4. The cell culture system of clause 3, wherein the patterning rail includes a flow blocking element configured to separate the fluid in each of the one or more regions.
5. The cell culture system of any of clauses 1-4, wherein the patterning rail detachably couples to the array using one clip or two clips.
6. A patterning rail configured to attach to a cell culture system, the patterning rail including a first port and a second port connected by an open microfluidic channel.
7. The patterning rail of clause 6, wherein the cell culture system includes:
   an array including a first post and a second post, the first post being configured to couple to the first port and the second post being configured to couple to the second port; and
   a cell culture vessel configured to receive the array.
8. The patterning rail of clause 7, wherein the patterning rail is configured to attach to the array with a holding clip or two holding clips.
9. The patterning rail of any of clauses 6-8, including:
   a flow blocking element configured to prevent the flow of a fluid in the open microfluidic channel; and
   a plurality of regions, wherein each of the plurality of region includes a volume within the open microfluidic channel such that fluid within each region is separated by the flow blocking element.
10. The patterning rail of any of clauses 6-9, further including an inner channel disposed in the open microfluidic channel, wherein the inner channel includes a crosslinked polymer.
11. A method including:
   coupling a patterning rail to a post array, the patterning rail including a first port, a second port, and an open microfluidic channel;
   receiving a cell composition into the first port of the patterning rail;
   culturing the cell composition in a cell culture vessel; and
   uncoupling the patterning rail from the post array, thereby preparing a suspended tissue.
12. The method of clause 11, wherein the cell composition includes cells and a hydrogel, wherein the cells are selected from fibroblasts, epithelial cells, alpha cells, beta cells, delta cells, cardiomyocytes, endothelial cells, hepatocytes, hepatic stellate cells, Kupffer cells (KCs), liver sinusoidal endothelial cells (LSECs), endocrine cells, myoblasts, chondrocytes, neurons, osteoblasts, osteoclasts, kidney cells, induced pluripotent stem cells, embryonic stem cells, mesenchymal stem cells, hematopoietic stem cells, tenocytes, and myocytes, and wherein the hydrogel includes a material selected from gelatin, fibrin, collagen, chitosan, fibronectin, laminin, alginate, Matrigel, hyaluronic acid, polyethylene glycol (PEG), and polyacrylamide.
13. The method of clause 12, wherein a thickness of the hydrogel is in a range of about 100 μm to about 10 mm, and/or
   wherein the cell composition further includes growth factors.
14. The method of any of clauses 11-13, further including at least one of:
   administering a second cell composition to the second port; or
   administering a third cell composition to a region of the open microfluidic channel.
15. The method of any of clauses 11-14, wherein the cell culture vessel includes culture media, and wherein the culturing includes:
   immersing the patterning rail in the culture media; and
   incubating the cell culture vessel in a cell culture environment including:
   a temperature of about 35° C. to about 40° C., and/or
   a CO₂ level of about 4% to about 10%.
16. The method of any of clauses 11-15, wherein uncoupling the patterning rail includes degrading an inner channel, the inner channel being disposed in the open microfluidic channel and including a crosslinked polymer.
17. The method of any of clauses 11-16, further including gelling the cell composition.
18. The method of any of clauses 11-17, further including:
   based on uncoupling the patterning rail from array, culturing the suspended tissue in the cell culture vessel; and/or
   repeating the method such that a subsequent layer forms over the suspended tissue.
19. The method of any of clauses 11-18, further including encapsulating the suspended tissue within an engineered tissue.
20. The method of any of clauses 11-19, wherein the suspended tissue includes
   cells selected from fibroblasts, epithelial cells, alpha cells, beta cells, delta cells, cardiomyocytes, endothelial cells, hepatocytes, hepatic stellate cells, Kupffer cells (KCs), liver sinusoidal endothelial cells (LSECs), endocrine cells, myoblasts, chondrocytes, neurons, osteoblasts, osteoclasts, kidney cells, induced pluripotent stem cells, embryonic stem cells, mesenchymal stem cells, hematopoietic stem cells, tenocytes, and myocytes; or
      artificial meat.

### Experimental Example 1

Utilizing Suspended Tissue Open Microfluidic Patterning (STOMP) for the Advancement of Tissue Engineering. Experimental Example 1 provides specific examples of engineered tissues and techniques for generating the engineered tissues. However, implementations of the present disclosure are not limited to the specific examples described with reference to Experimental Example 1.

What is claimed is:

1. A cell culture system comprising:
a patterning rail comprising a first port and a second port connected by an open microfluidic channel;
an array comprising a first post and a second post, the first post being configured to couple to the first port and the second post being configured to couple to the second port; and
an attachment mechanism configured to removably attach the patterning rail to the array; and
a cell culture vessel configured to receive the array.

2. The cell culture system of claim 1, wherein the patterning rail comprises a polymer, glass, plastic, or metal, and
wherein the first post and/or the second post comprise glass, plastic, metal, silicone, flexible silicone, polydimethylsiloxane (PDMS), or a glass rod.

3. The cell culture system of claim 1, wherein the open microfluidic channel comprises:
walls separated by a space; and
one or more regions, wherein each of the one or more regions comprises a volume within the open microfluidic channel such that fluid within each region is separated.

4. The cell culture system of claim 3, wherein the patterning rail comprises a flow blocking element configured to separate the fluid in each of the one or more regions.

5. The cell culture system of claim 1, wherein the patterning rail detachably couples to the array using one clip or two clips.

6. A patterning rail configured to attach to a cell culture system, the patterning rail comprising a first port and a second port connected by an open microfluidic channel.

7. The patterning rail of claim 6, wherein the cell culture system comprises:
an array comprising a first post and a second post, the first post being configured to couple to the first port and the second post being configured to couple to the second port; and
a cell culture vessel configured to receive the array.

8. The patterning rail of claim 7, wherein the patterning rail is configured to attach to the array with a holding clip or two holding clips.

9. The patterning rail of claim 6, comprising:
a flow blocking element configured to prevent the flow of a fluid in the open microfluidic channel; and
a plurality of regions, wherein each of the plurality of region comprises a volume within the open microfluidic channel such that fluid within each region is separated by the flow blocking element.

10. The patterning rail of claim 6, further comprising an inner channel disposed in the open microfluidic channel, wherein the inner channel comprises a crosslinked polymer.

11. A method comprising:
coupling a patterning rail to a post array, the patterning rail comprising a first port, a second port, and an open microfluidic channel;
receiving a cell composition into the first port of the patterning rail;
culturing the cell composition in a cell culture vessel; and
uncoupling the patterning rail from the post array, thereby preparing a suspended tissue.

12. The method of claim 11, wherein the cell composition comprises cells and a hydrogel, wherein the cells are selected from fibroblasts, epithelial cells, alpha cells, beta cells, delta cells, cardiomyocytes, endothelial cells, hepatocytes, hepatic stellate cells, Kupffer cells (KCs), liver sinusoidal endothelial cells (LSECs), endocrine cells, myoblasts, chondrocytes, neurons, osteoblasts, osteoclasts, kidney cells, induced pluripotent stem cells, embryonic stem cells, mesenchymal stem cells, hematopoietic stem cells, tenocytes, and myocytes, and
wherein the hydrogel comprises a material selected from gelatin, fibrin, collagen, chitosan, fibronectin, laminin, alginate, Matrigel, hyaluronic acid, polyethylene glycol (PEG), and polyacrylamide.

13. The method of claim 12, wherein a thickness of the hydrogel is in a range of about 100 μm to about 10 mm, and/or
wherein the cell composition further comprises growth factors.

14. The method of claim 11, further comprising at least one of:
administering a second cell composition to the second port; or
administering a third cell composition to a region of the open microfluidic channel.

15. The method of claim 11, wherein the cell culture vessel includes culture media, and wherein the culturing comprises:
immersing the patterning rail in the culture media; and
incubating the cell culture vessel in a cell culture environment comprising:
a temperature of about 35° C. to about 40° C., and/or
a CO₂ level of about 4% to about 10%.

16. The method of claim 11, wherein uncoupling the patterning rail comprises degrading an inner channel, the inner channel being disposed in the open microfluidic channel and comprising a crosslinked polymer.

17. The method of claim 11, further comprising gelling the cell composition.

18. The method of claim 11, further comprising:
based on uncoupling the patterning rail from array, culturing the suspended tissue in the cell culture vessel; and/or
repeating the method such that a subsequent layer forms over the suspended tissue.

19. The method of claim 11, further comprising encapsulating the suspended tissue within an engineered tissue.

20. The method of claim 11, wherein the suspended tissue comprises
cells selected from fibroblasts, epithelial cells, alpha cells, beta cells, delta cells, cardiomyocytes, endothelial cells, hepatocytes, hepatic stellate cells, Kupffer cells (KCs), liver sinusoidal endothelial cells (LSECs), endocrine cells, myoblasts, chondrocytes, neurons, osteoblasts, osteoclasts, kidney cells, induced pluripotent stem cells, embryonic stem cells, mesenchymal stem cells, hematopoietic stem cells, tenocytes, and myocytes; or
artificial meat.
